(19) United States
(12) Patent Application Publication
(10) Pub. No.: US 2015/0164755 A1
(43) Pub. Date: Jun. 18, 2015

(54) Use Of N-Methyl-N-Acylglucamines As Cold Stabilizers In Surfactant Solutions

(71) Applicants: Peter Klug, Grossostheim (DE); Carina Mildner, Frankfurt am Main (DE)

(72) Inventors: Peter Klug, Grossostheim (DE); Carina Mildner, Frankfurt am Main (DE)

(73) Assignee: Clariant Finance (BVI0 Limited, Tortola (VG)

(21) Appl. No.: 14/402,996

(22) PCT Filed: May 29, 2013
(86) PCT No.: PCT/EP2013/061046

§ 371 Date: Nov. 21, 2014

(30) Foreign Application Priority Data
May 30, 2012 (DE) 10 2012 010 700.5

(51) Int. Cl. A61K 8/42; A61Q 19/10; A61Q 5/02; A61K 8/46; A61K 8/44

(52) U.S. Cl. CPC A61K 8/42; A61K 8/463; A61K 8/44; A61Q 5/02; A61Q 19/10; A61K 2800/5922

(57) ABSTRACT

The invention relates to the use of N-methyl-N-acylglucamines as cold stabilizers in aqueous surfactant solutions, wherein at least 90 wt.-% of the N-methyl-N-acylglucamines have a C₈-acyl or C₁₀-acyl group. The invention further relates to compositions containing (a) N-methyl-N-acylglucamines, wherein at least 90 wt.-% of the N-methyl-N-acylglucamines have a C₈-acyl or C₁₀-acyl group as component (A), (b) one or more anionic surfactants as component (B), (c) one or more betaine surfactants as component (C), (d) optionally additional surfactants as component (D), (e) water as component (E), and (f) optionally additional additives as component (F).

The invention relates to the use of N-methyl-N-acylglucamines as cold stabilizers in aqueous surfactant solutions.

It is known that short-chain sugar surfactants can be used as solubilizers in surface-active products.

In the production of liquid surface-active products such as hair shampoos or machine dishwashing or manual dishwashing products, it is frequently a problem that the surfactant ingredients do not have sufficient water solubility and, especially in the presence of salts, cause cloudiness and form several phases. For solubilizers of the surfactant ingredients in surface-active products such as washing, rinsing and cleaning products, and in cosmetic or pharmaceutical formulations, WO 96/14374 discloses N-alkyl-N-polyhydroxyalkylcarboxamides of the formula

R²CO—NR³—[Z]

in which R²CO is an aliphatic acyl radical having 1 to 8 carbon atoms, R³ is hydrogen or an alkyl or hydroxyalkyl radical having 1 to 8 carbon atoms and [Z] is a polyhydroxyalkyl radical having 3 to 12 carbon atoms and 3 to 10 OH groups. Preference is given to carboxyl-N-alkylglucamines in which R²CO is the acyl radical of formic acid, acetic acid, propionic acid, butyric acid or caproic acid, and the alkyl radical R³ is methyl or octyl. In the examples, acetyloxy-N-octylglucamine, butyryloxy-N-octylglucamine and caproyloxy-N-methylglucamine are used as solubilizers for a mixture comprising sodium oleinsulfonate, coconut fatty acid alcohol ether sulfate sodium salt and coconut fatty acid triethanolammonium salt.

WO 95/17880 discloses a hair shampoo composition comprising alkyl glycol ether sulfates and alkyl sulfates, and also polyhydroxyalkyl fatty acid amides. One alkyl glycol ether sulfate mentioned is lauryl triethylene glycol ether sulfate; one alkyl sulfate mentioned is lauryl sulfate. Compounds listed as polyhydroxyalkyl fatty acid amides are those of the general formula

R²—CO—NR¹—Z

where R¹ is preferably C₁-C₄-alkyl, especially methyl, R² is preferably straight-chain C₇-C₁₉-alkyl or -alkenyl, especially straight-chain C₁₁-C₁₆-alkyl or -alkenyl, and Z is especially 1-deoxyglucityl, 2-deoxyfructityl, 1-deoxymaltityl, 1-deoxylactityl, 1-deoxygalactityl, 1-deoxymannityl or 1-deoxymaltotriothityl. The examples disclose hair shampoo compositions comprising ammonium lauryl sulfate, ammonium lauryl triethylene glycol sulfate and lauryl-N-methylglucamine.

In the case of aqueous surfactant solutions comprising linear alkyl sulfates and/or alkyl ether sulfates on the one hand and betaine surfactants on the other hand, the viscosity decreases with falling temperature. This can be attributed to precipitation of surfactants out of the solution at low temperatures. This effect is known in practice as the “sports bag effect” and is unwanted, since personal care and hair washing products become as mobile as water when stored under cold conditions and no longer have the desired handling profile.

It is an object of the invention to provide a stabilizer for improving the cold stability of aqueous surfactant solutions.

The object is achieved by the use of N-methyl-N-acylglucamines where at least 90% by weight of the N-methyl-N-acylglucamines contain a C₈-acyl or C₁₀-acyl group as cold stabilizers in aqueous surfactant solutions.

N-Methyl-N-acylglucamines have the general formula (I)

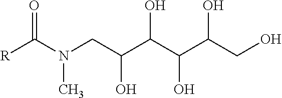

in which R is a corresponding alkyl radical or a mono- or polyunsaturated alkenyl radical, i.e. a C₇- or C₉-alkyl radical or a mono- or polyunsaturated alkenyl radical in the case of the C₈- or C₁₀-acylglucamines.

It has been found that, in the presence of the N-methyl-N—C₈-C₁₀-acylglucamines used in accordance with the invention, the viscosity of the aqueous surfactant solutions is still surprisingly high, even at low temperatures.

The N-methyl-N-acylglucamines used in accordance with the invention consist to an extent of at least 90% by weight of N-methyl-N-acylglucamines containing a C₈- or C₁₀-acyl group. More preferably, the proportion of N-methyl-N-acylglucamines containing a C₈ or C₁₀-acyl group is at least 95% by weight. In addition, the N-methyl-N-acylglucamines used as solubilizers in accordance with the invention contain small proportions of N-methyl-N-acylglucamines derived from short-chain and/or long-chain fatty acids, especially those containing C₁-C₄-acyl, C₆-, C₁₂-, C₁₄-, C₁₆-, C₁₈- and/or C₂₀-acyl.

The N-methyl-N-acylglucamines can, as described in EP 0 550 637 B1, be prepared by reacting the corresponding fatty acid esters or fatty acid ester mixtures with N-methylglucamine in the presence of a solvent having hydroxyl groups or alkoxy groups. Suitable solvents are, for example, C₁-C₄ monoalcohols, ethylene glycol, propylene glycol, glycerol and alkoxylated alcohols. Preference is given to 1,2-propylene glycol. N-Methylglucamine can, as likewise described in EP 0 550 637 A1, be obtained by reductive amination of glucose with methylamine.

Suitable fatty acid esters which are reacted with the N-methylglucamines to give N-methyl-N-acylglucamines are generally the methyl esters, which are obtained by transesterification from natural fats and oils, for example the triglycerides.

Suitable raw materials for the preparation of the fatty acid methyl esters are, for example, coconut oil or palm oil.

In general, the aqueous surfactant solutions comprise both anionic surfactants and betaine surfactants.

In a preferred embodiment of the invention, the aqueous surfactant solutions comprise one or more anionic surfactants from the group of the alkyl sulfates and alkyl ether sulfates.

Preferred alkyl sulfates are the C₈-C₂₀-alkyl sulfates, especially the linear C₈-C₂₀-alkyl sulfates in the form of their sodium, potassium or ammonium salts. Examples of alkyl sulfates are lauryl sulfate, cocoalkyl sulfate and tallowalkyl sulfate. Particular preference is given to lauryl sulfate.

Preferred alkyl ether sulfates are the C₈-C₂₀-alkyl ether sulfates, particular preference being given to the linear C₈-C₂₀-alkyl ether sulfates, especially the alkyl glycol ether sulfates derived from the ethoxylated fatty alcohols, in the form of their sodium, potassium or ammonium salts. Examples of alkyl ether sulfates are lauryl ether sulfate, cocoalkyl ether sulfate and tallowalkyl ether sulfate. Examples of glycol ether sulfates are lauryl triethylene glycol ether sulfate, cocoalkyl triethylene glycol ether sulfate and tallowalkyl hexaethylene glycol ether sulfate. Lauryl glycol ether sulfate is especially preferred, for example lauryl diethylene glycol ether sulfate and lauryl triethylene glycol ether sulfate, especially in the form of the sodium salts.

Preferably, the aqueous surfactant solutions comprise, as well as the anionic surfactant, a betaine surfactant.

Betaine surfactants contain, in the same molecule, a cationic group, especially an ammonium group, and an anionic group, which may be a carboxylate group, sulfate group or sulfonate group. Suitable betaines are alkyl betaines such as cocobetaine of fatty acid alkylamidopropyl betaines, for example cocoacylamidopropyl dimethyl betaine, C₁₂-C₁₈ dimethylamino-hexanoates or C₁₀-C₁₈ acylamidopropane dimethyl betaines.

In a preferred embodiment of the invention, the aqueous surfactant solutions comprise one or more amidopropyl betaines of the general formula (I)

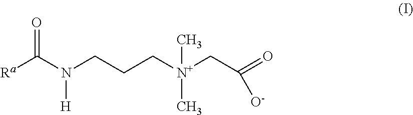

in which R^{a} is a linear or branched saturated C₇-C₂₁-alkyl group or a linear or branched mono- or polyunsaturated C₇-C₂₁-alkenyl group.

In a further preferred embodiment of the invention, the surfactant solutions comprise one or more betaines of the formula (II)

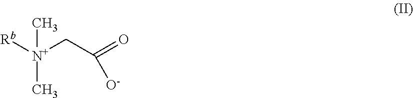

in which R^{b} is a linear or branched saturated C₈-C₂₂-alkyl group or a linear or branched mono- or polyunsaturated C₈-C₂₂-alkenyl group.

In a further preferred embodiment of the invention, the surfactant solutions comprise one or more sulfo betaines of the formula (III)

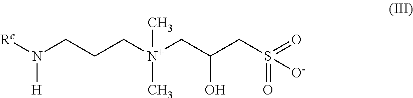

in which R^{c} is a linear or branched saturated C₈-C₂₂-alkyl group or a linear or branched mono- or polyunsaturated C₈-C₂₂-alkenyl group.

More preferably, the surfactant solutions comprise one or more betaine surfactants selected from the group of the compounds consisting of the amidopropyl betaines of the formula (I), the betaines of the formula (II) and the sulfo betaines of the formula (III).

In an especially preferred embodiment of the invention, the surfactant solutions comprise one or more betaine surfactants selected from the amidopropyl betaines of the formula (I).

In a further especially preferred embodiment of the invention, the surfactant solutions comprise one or more betaine surfactants selected from the betaines of the formula (II).

In a further especially preferred embodiment of the invention, the surfactant solutions comprise one or more betaine surfactants selected from the sulfo betaines of the formula (III).

Preferably, the R^{a} radical in the one or more amidopropyl betaines of the formula (I) is a linear or branched saturated C₇-C₁₇-alkyl group. Among the linear and branched saturated alkyl groups R², preference is given to the linear saturated alkyl groups.

More preferably, the amidopropyl betaines of the formula (I) are cocamidopropyl betaines.

Preferably, the R^{b} radical in the one or more betaines of the formula (II) is a linear or branched saturated C₈-C₁₈-alkyl group and more preferably a linear or branched saturated C₁₂-C₁₈-alkyl group. Among the linear and branched saturated alkyl groups R^{b}, preference is given to the linear saturated alkyl groups.

Preferably, the R^{c} radical in the one or more sulfo betaines of the formula (III) is a linear or branched saturated C₈-C₁₈-alkyl group and more preferably a linear or branched saturated C₁₂-C₁₈-alkyl group. Among the linear and branched saturated alkyl groups R^{c}, preference is given to the linear saturated alkyl groups.

More preferably, the aqueous surfactant solutions comprise amidopropyl betaines of the formula (I) and/or alkyl betaines of the formula (II).

The invention also provides compositions comprising
(a) N-methyl-N-acylglucamines where at least 90% by weight of the N-methyl-N-acylglucamines contain a C₈-acyl or C₁₀-acyl group as component (A),
(b) anionic surfactants as component (B),
(c) betaine surfactants as component (C),
(d) optionally further surfactants as component (D),
(e) water as component (E), and
(f) optionally further additives, such as preservatives, fragrances, dyes and superfatting agents, as component (F).

In general, the compositions comprise
(a) 0.01% to 5.0% by weight, preferably 0.1% to 3.0% by weight, of component (A),
(b) 1.0% to 20.0% by weight, preferably 5.0% to 15% by weight, of component (B),
(c) 0.1% to 10.0% by weight, preferably 1.0% to 10.0% by weight, of component (C),
(d) 0% to 5.0% by weight, preferably 0% to 3.0% by weight, of component (D),
(e) 55.0% to 98.89% by weight, preferably 75% to 95% by weight, of component (E),
(f) 0% to 5.0% by weight, preferably 0% to 2.0% by weight, of component (F).

Preferably, the inventive cosmetic compositions comprise the above-described alkyl sulfates and/or alkyl ether sulfates and betaine surfactants.

Optional further surfactants (D) may be cationic, nonionic or amphoteric surfactants.

Suitable cationic surfactants are substituted or unsubstituted, straight-chain or branched quaternary ammonium salts of the R¹N(CH₃)₃X, R¹R²N(CH₃)₂X, R¹R²R³N(CH₃)X or R¹R²R³R⁴NX type. The R¹, R², R³ and R⁴ radicals may preferably each independently be unsubstituted alkyl having a chain length between 8 and 24 carbon atoms, especially between 10 and 18 carbon atoms, hydroxyalkyl having 1 to 4 carbon atoms, phenyl, C₂- to C₁₈-alkenyl, C₇- to C₂₄-aralkyl, (C₂H₄O)ₓH where x is from 1 to 3, alkyl radicals containing one or more ester groups, or cyclic quaternary ammonium salts. X is a suitable anion. Preference is given to (C₈-C₂₂)-alkyltrimethylammonium chloride or bromide, more preferably cetyl-trimethylammonium chloride or bromide, di-(C₈-C₂₂)-alkyldimethyl-ammonium chloride or bromide, (C₈-C₂₂)-alkyldimethylbenzylammonium chloride or bromide, (C₈-C₂₂)-alkyldimethylhydroxyethylammonium chloride, phosphate, sulfate, lactate, more preferably distearyldimethylammonium chloride, di-(C₈-C₂₂)-alkylamidopropyltrimethyl-ammonium chloride and methosulfate.

Examples of useful nonionic surfactants include the following compounds:
Polyethylene oxide, polypropylene oxide and polybutylene oxide condensates of alkylphenols. These compounds comprise the condensation products of alkylphenols having a C₆- to C₂₀-alkyl group which may be either linear or branched with alkylene oxides. These surfactants are referred to as alkylphenol alkoxylates, e.g. alkylphenol ethoxylates.
Condensation products of aliphatic alcohols with 1 to 25 mol of ethylene oxide. The alkyl or alkenyl chain of the aliphatic alcohols may be linear or branched, primary or secondary, and contains generally 8 to 22 carbon atoms. Particular preference is given to the condensation products of C₁₀ to C₂₀ alcohols with 2 to 18 mol of ethylene oxide per mole of alcohol. The alcohol ethoxylates may have a narrow (“narrow range ethoxylates”) or a broad homolog distribution of the ethylene oxide (“broad range ethoxylates”). Examples of commercially available nonionic surfactants of this type are Tergitol® 15-S-9 (condensation product of a linear secondary C₁₁-C₁₅ alcohol with 9 mol of ethylene oxide), Tergitol® 24-L-NMW (condensation product of a linear primary C₁₂-C₁₄ alcohol with 6 mol of ethylene oxide, having narrow molar mass distribution). This product class likewise includes the Genapol® brands from Clariant.
Condensation products of ethylene oxide with a hydrophobic basis, formed by condensation of propylene oxide with propylene glycol. The hydrophobic moiety of these compounds preferably has a molecular weight between 1500 and 1800. The addition of ethylene oxide onto this hydrophobic moiety leads to an improvement in the water solubility. The product is liquid up to a polyoxyethylene content of about 50% of the total weight of the condensation product, which corresponds to a condensation with up to about 40 mol of ethylene oxide. Commercially available examples of this product class are the Pluronic® brands from BASF and the Genapol® PF brands from Clariant.
Condensation products of ethylene oxide with a reaction product of propylene oxide and ethylenediamine. The hydrophobic unit of these compounds consists of the reaction product of ethylenediamine with excess propylene oxide and generally has a molecular weight of 2500 to 3000. Ethylene oxide is added onto this hydrophobic unit up to a content of 40 to 80% by weight of polyoxyethylene and a molecular weight of 5000 to 11 000. Commercially available examples of this compound class are the Tetronic® brands from BASF and the Genapol® PN brands from Clariant.

Further suitable nonionic surfactants are alkyl and alkenyl oligoglycosides and fatty acid polyglycol esters or fatty amine polyglycol esters each having 8 to 20 and preferably 12 to 18 carbon atoms in the fatty alkyl radical, alkyl oligoglycosides, alkenyl oligoglycosides and fatty acid N-alkylglucamides.

In addition, the inventive compositions may comprise amphoteric surfactants. These may be described as derivatives of long-chain secondary or tertiary amines which have an alkyl group having 8 to 18 carbon atoms and in which a further group is substituted by an anionic group which imparts water solubility, for example by a carboxyl, sulfate or sulfonate group. Preferred amphosurfactants are N—(C₁₂-C₁₈)alkyl β-aminopropionates and N—(C₁₂-C₁₈)alkyl β-iminodipropionates in the form of alkali metal and mono-, di- and trialkylammonium salts. Suitable further surfactants are also amine oxides. These are oxides of tertiary amines having a long-chain group of 8 to 18 carbon atoms and two usually short-chain alkyl groups having 1 to 4 carbon atoms. Preference is given here, for example, to the C₁₀- to C₁₈-alkyldimethylamine oxides and fatty acid amidoalkyl dimethylamine oxides.

Further additives (F) are, for example, preservatives, fragrances, dyes and refatting agents.

Suitable preservatives are the preservatives listed in the relevant annex of the European cosmetics legislation, for example phenoxyethanol, benzyl alcohol, parabens, benzoic acid and sorbic acid; a particularly suitable example is 1,3-bis(hydroxymethyl)-5,5-dimethylimidazolidine-2,4-dione (Nipaguard® DMDMH).

The amount of the preservatives in the inventive compositions is generally from 0% to 2.0% by weight, based on the total weight of the finished compositions.

Fragrances used may be individual odorant compounds, for example the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethole, citronellol, eugenol, geraniol, linalool, phenylethyl alcohol and terpineol, and the hydrocarbons include primarily the terpenes and balsams. Preference is given to using mixtures of different odorants which together produce a pleasing scent note.

Fragrances used may also be natural odorant mixtures, as obtainable from vegetable or animal sources, for example pine oil, citrus oil, jasmine oil, lily oil, rose oil or ylang-ylang oil. Essential oils of relatively low volatility, which are usually used as aromatic components, are also suitable as perfume oils, for example sage oil, chamomile oil, clove oil, melissa oil, mint oil, cinnamon leaf oil, linden blossom oil, juniperberry oil, vetiver oil, olibanum oil, galbanum oil and ladanum oil.

The amount of the fragrances in the inventive compositions is generally from 0% to 2% by weight, based on the total weight of the finished compositions.

Superfatting agents used may preferably be lanolin and lecithin, nonethoxylated and polyethoxylated or acylated lanolin derivatives and lecithin derivatives, polyol fatty acid esters, mono-, di- and triglycerides and/or fatty acid alkanolamides, the latter simultaneously serving as foam stabilizers, which are preferably used in amounts of 0.01% to 10.0% by weight, more preferably of 0.1% to 5.0% by weight and especially preferably of 0.5% to 3.0% by weight.

The dyes and color pigments present in the inventive compositions, both organic and inorganic dyes, may be selected from the corresponding positive list in the cosmetics regulations, or the EU list of cosmetic colorants. Also used advantageously are pearlescent pigments, for example pearl essence (guanine/hypoxanthine mixed crystals from fish scales) and mother of pearl (ground mussel shells), monocrystalline pearlescent pigments, for example bismuth oxychloride (BiOCl), layer-substrate pigments, for example mica/metal oxide, silver-white pearlescent pigments composed of TiO₂, interference pigments (TiO₂, different layer thickness), color luster pigments (Fe₂O₃) and combination pigments (TiO₂/Fe₂O₃, TiO₂/Cr₂O₃, TiO₂/prussian blue, TiO₂/carmine).

In a preferred embodiment of the invention, the inventive compositions are in the form of products for hair washing and skin cleansing, such as hair shampoos, shower gels, hand soaps and face cleansers.

The invention is illustrated in detail by the examples which follow.

### EXAMPLES

### Examples 1 and 2 and Comparative Examples 1 to 4

The N-acyl-N-methylglucamines described hereinafter were prepared according to EP 0 550 637 from the corresponding fatty acid methyl esters and N-methylglucamine in the presence of 1,2-propylene glycol as solvent, and were obtained in solid form consisting of active substance and 1,2-propylene glycol (all figures in % by weight).

TABLE 1

| | Methyl | Active | 1,2-Propylene | Melting |
|---|---|---|---|---|
| Example | ester | substance (%) | glycol (%) | point |
| Comparative | C12/14 | 90 | 10 | 85 |
| example 1 | | | | |
| 1 | C8/10 | 90 | 10 | 50 |

The viscosities were measured with a Brookfield DV II viscometer, the spindles from the RV spindle set at 20 revolutions/minute and 20° C. Spindles 1 to 7 from the RV spindle set were used. Under these measurement conditions, spindle 1 was chosen for viscosities of not more than 500 mPa·s, spindle 2 for viscosities of not more than 1000 mPa·s, spindle 3 for viscosities of not more than 5000 mPa·s, spindle 4 for viscosities of not more than 10 000 mPa·s, spindle 5 for viscosities of not more than 20 000 mPa·s, spindle 6 for viscosities of not more than 50 000 mPa·s and spindle 7 for viscosities of not more than 200 000 mPa·s.

Surfactant solutions consisting of sodium lauryl ether sulfate (Genapol LRO liq., Clariant), cocoamidopropyl betaine (Genagen CAB 818, Clariant) were prepared in a ratio of 7:3 with total active substance 15% by weight, and adjusted to a uniform viscosity of about 5000 mPas by addition of sodium chloride.

In further experiments, these surfactant solutions were admixed with 1% by weight of additional sugar surfactant, and likewise adjusted to viscosity about 5000 mPas with salt.

The viscosities were ascertained at 20° C., and then the example formulations were cooled to 4° C. and the viscosity was measured again.

The extent of cold stability was ascertained as the reduction in viscosity on cooling in %.

TABLE 2

| | | | | | | Amount |
|---|---|---|---|---|---|---|
| | | | | | | of salt |
| | | | | Reduction | Appearance | required |
| | | | | in | of the | for 5000 |
| | Sugar | Viscosity | Viscosity | viscosity | solution at | mPas |
| Example | surfactant | at 20° C. | at 4° C. | (%) | 4° C. | (%) |
| Example 2 | Example 1 | 4780 | 2400 | −50 | clear | 1.2 |
| Comparative | Comparative | 4800 | 1330 | −72 | cloudy | 0.50 |
| example 2 | example 1 | | | | | |
| Comparative | Plantacare | 5150 | 1580 | −69 | cloudy | 0.80 |
| example 3 | 818 (coco- | | | | | |
| | glucoside) | | | | | |
| Comparative | none | 4800 | 1410 | −71 | cloudy | 1.0 |
| example 4 | | | | | | |

As apparent from the above table, inventive example 1 shows a significantly smaller reduction in viscosity on cooling of the surfactant solution, and the surfactant solution additionally remains clear, in contrast to the comparative examples.

1. The use of N-methyl-N-acylglucamines as cold stabilizers in aqueous surfactant solutions, where at least 90% by weight of the N-methyl-N-acylglucamines contain a C₈-acyl or C₁₀-acyl group.

2. The use as claimed in claim 1, wherein the aqueous surfactant solutions comprise an alkyl sulfate and/or an alkyl ether sulfate as anionic surfactant and a betaine surfactant.

3. The use as claimed in claim 1 or 2, wherein the aqueous surfactant solutions comprise a linear C₈-C₂₀-alkyl sulfate and/or a linear C₈-C₂₀-alkyl ether sulfate.

4. The use as claimed in claim 3, wherein the aqueous surfactant solutions comprise lauryl sulfate and/or a lauryl ether sulfate.

5. The use as claimed in any of claims 1 to 4, wherein the aqueous surfactant solutions comprise an acylamidopropyl betaine or an alkyl betaine.

6. A composition comprising
(a) N-methyl-N-acylglucamines where at least 90% by weight of the N-methyl-N-acylglucamines contain a C₈-acyl or C₁₀-acyl group as component (A),
(b) one or more anionic surfactants as component (B),
(c) one or more betaine surfactants as component (C),
(d) optionally further surfactants as component (D),
(e) water as component (E),
(f) optionally further additives as component (F).

7. A composition comprising
(a) 0.01% to 5.0% by weight of component (A),
(b) 1.0% to 20.0% by weight of component (B),
(c) 0.1% to 10.0% by weight of component (C),
(d) 0% to 5.0% by weight of component (D),
(e) 55.0% to 98.89% by weight of component (E),
(f) 0% to 5.0% by weight of component (F).

8. The composition as claimed in claim 6 or 7 in the form of a hair shampoo, shower gel, hand soap or face cleanser.
